(19) United States
(12) Patent Application Publication
(10) Pub. No.: US 2006/0224136 A1
(43) Pub. Date: Oct. 5, 2006

(54) Cloth menstrual pad with hemp core

(76) Inventor: Christina Marie Gabbard Martinez, Clemmons, NC (US)

Correspondence Address: Christina Marie Gabbard, 284 Cinnamon Way, Clemmons, NC, 27012, US

(21) Appl. No.: 11/358,850

(22) Filed: Feb. 21, 2006

Related U.S. Application Data
(60) Provisional application No. 60/668,882, filed on Mar. 31, 2005.

(51) Int. Cl. A61F 13/15

(52) U.S. Cl. 604/385.15; 604/367

(57) ABSTRACT

Washable reusable cloth menstrual pad with Hemp core.

### FIELD OF THE INVENTION

The present invention relates to menstrual pads and, more particularly, washable and reusable cloth menstrual pad with Hemp core.

Washable, reusable cloth menstrual pad to be worn during menses, normally occurring in no-pregnant women about every four weeks, from menarche to menopause. To be worn during times of mild urinary incontinence. Menstrual pad comfort is largely a matter of peace of mind, meaning that one feels good about the environmental effect of the product, fluid impervious nature, and its overall comfort-ability. Reusable cloth menstrual pads provide the users an environmentally conscious alternative, leak-proof discreet protection and a comfortable slim-line design.

### BACKGROUND OF THE INVENTION

Readily available disposable menstrual pads can cause negative effects on the environment, skin irritation and chaffing from bleaching and adhesives and unnecessary wetness against the users skin. Menstruation is big business. Of the 240 million Americans, over 125 million are women all of which will experience menstruation from menarche at approximately age 12 to menopause at approximately 50. During those 28 years, with the exceptions of pregnancies and skipped periods, they will deal with it 12 times a year and buy 360 boxes of tampons or pads in their lives. This generates constant revenue of approximately $1000 per woman equaling $1.25 billion dollars in the US alone.

Menstrual pad comfort is largely a matter of peace of mind, meaning that one feels good about the environmental effect of the product and it's overall comfortability. Women want a natural, ecb-friendly, easy to use device that is functional, practical and esthetically pleasing.

Tampons and Sanitary Pads.

Readily available disposable sanitary pads and tampons can cause negative effects on the environment, skin irritation and chaffing from bleaching and adhesives and unnecessary wetness against the users skin. Menstrual pad comfort is largely a matter of peace of mind, meaning that one feels good about the environmental effect of the product and it's overall comfortability. Reusable cloth menstrual pads provide the users a natural, environmentally conscious alternative, discreet protection and a comfortable slim line design.

Toxic chemicals are contained in disposable products, which are bleached with chlorine compounds. There is accumulating evidence that industrial uses of chlorine, including pulp and paper bleaching, releases toxic dioxins which bioaccumulate in the environment causing serious harm to wildlife.

Tampons and pads are often over packaged and cause enormous waste. The average woman will go through about 10,000 pads or tampons in her life not to mention the millions of unnecessary plastic tampon applicators which wash up on beaches around the world and fill up landfills.

In addition to this ecological damage, there are also personal dangers caused by sanitary product use. Dioxin, a by-product of the chlorine bleaching process, has a number of serious health impacts: the effects of shredding rayon fibers from tampons in women's vaginas, the probable link between dioxin and endometriosis, the possibility of cervical cancer being linked to prolonged tampon use over many years, toxic shock syndrome, headaches and so on.

All the major brands (Tampax, Playtex, O.B.) use the chlorine bleaching process to whiten their products. Aside from leaving behind minute quantities of toxic dioxins in the product and releasing dioxins into our rivers and waterways, there is absolutely no logical reason for bleaching sanitary products whiter than white. Tampons are not sterile. All the major brands contain rayon that is a pulp product that can only be made through a chlorine or chlorinated compound bleaching process. Fiber loss from rayon has been traced as the probable cause of Toxic Shock Syndrome and has been shown to damage a woman's vagina by causing ulceration and a peeling of the mucous membrane

It is therefore an object of the invention to provide an easily washable and reusable device.

It is therefore an object of the invention to provide an absorbent Hemp Core positioned between the top Bodyside Fabric and the bottom Backside Fabric.

It is therefore an object of the invention to provide a Leak Guard, a liquid impervious layer to ensure no leakage or soilage from penetrating outer garments.

It is therefore an object of the invention to provide a natural organic or nonorganic Bodyside Fabric for comfort and softness.

It is therefore an object of the invention to provide a cloth menstrual pad that is stabilized in original position securely around underwear by means of Resin Snap.

It is therefore an object of the present invention is to provide a natural cloth device that will not harm the wearer or the environment.

It is therefore an object of this invention to provide a slim line device that thoroughly and discreetly catches menstrual flow as well as urinary incontinence in a discreet manner.

It is intended that any other advantages and objects of the present invention that become apparent or obvious from the detailed description or illustrations contained herein are within the scope of the present invention.

### SUMMARY OF THE INVENTION

In accordance with the present invention, there is provided a Cloth Menstrual Pad with Hemp Core that is functional, practical and esthetically pleasing. Invention provides an easy to use, comfortable, leakproof and natural device to be worn during menses or urinary incontinence. Device can be worn by women of all age groups, from those entering menarche up and through menopause. Although constructed primarily for menstruation, the pad can be worn for other purposes such as post-sexual activity to collect drainage and discharge and can be use for mild urine incontinence by those suffering from urinary tract infection. Use of a hidden, gas permeable, liquid impermeable layer, which runs the entire length and width of the pad, prevents leakage or soilage from penetrating outer garments.

A 5 layer, hourglass with wings shaped body, which helps prevent pad from slipping around. The contoured center is also provided to provide a comfortable fit since it fits women's natural curves. The wings are provided as a means of securing the pad to the users undergarments by way of resin snap. Bodyside layer is organic cotton or non-organic woven material, two layer Hemp Core for absorption of fluids, single fluid impervious Leak Guard layer and Backside Fabric that is organic cotton or nonorganic woven material. All layers are sewn together around the perimeter, and then turned and topstitched, and then snap is applied.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a top detail (bodyside) view of a cloth menstrual pad with hemp core as it appears topside and open demonstrating the entire width and breadth of the pad. Shows the topstitching around perimeter of the pad, resin snap placement and hemp core topstitching

FIG. 2 is a snapped closed (underwear-side) view of a cloth menstrual pad with hemp core as it would appear snapped closed from the underside of the users undergarment. Shows topstitching, resin snap placement, bodyside fabric and backside fabric

FIG. 3 is a snapped closed carry pouch view of a cloth menstrual pad with hemp core as it appears snapped closed onto itself for convenient leak-proof transport when user is away from home. Shows resin snap placement, backside fabric and topstitching

FIG. 4 is an exploded detail view of a cloth menstrual pad with hemp core exploded view demonstrating layers of construction. Pieces are sewn together, wrong sides facing, then turned and topstitched. Resulting in a neat looking and slimline device

FIG. 5 is a snapped closed bodyside view of a cloth menstrual pad with hemp core as it would appear from the top, bodyside in the snapped closed position. Shows topstitching of hemp core and topstitching of perimeter of pad; and

FIG. 6 is a back perspective view of a cloth menstrual pad with hemp core. Shows entire width and breadth of the pad from the back.

For purposes of clarity and brevity, like elements and components will bear the same designations and numbering throughout the Figures.

### DESCRIPTION OF THE PREFERRED EMBODIMENT

Referring now descriptively to the drawings, the attached figures illustrate a reusable cloth menstrual pad with Hemp Core 22.

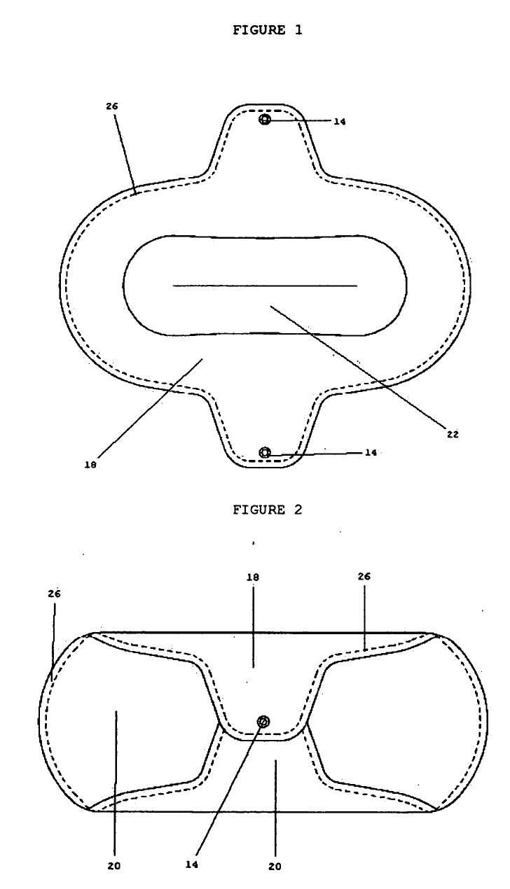

FIGS. 1 illustrates a Cloth Menstrual Pad with Hemp Core 22, showing the body side as it would appear to the wearer after being stabilized with Resin Snap 14, around user's undergarment. The Bodyside Fabric 18, is soft organic or nonorganic woven material. This illustration shows Hemp Core 22 Top-stitching 26 that aids in pulling menses or urine incontinence inward toward the hidden absorbent Hemp Core 22.

FIG. 2 illustrates pad stabilized with Resin Snap 14, around user's undergarment. View of a Reusable Cloth Menstrual Pad, as it would appear snapped closed with a single lightweight Resin Snap 14.

FIGS. 3 illustrates a Reusable Cloth Menstrual Pad with Hemp Core 22, open, bodyside up. Demonstrates entire breath and width of the pad. This illustration shows Hemp Core 22 Top-stitching 26 that aids in pulling the menstrual or urine flow inward toward the hidden absorbent Hemp Core 22.

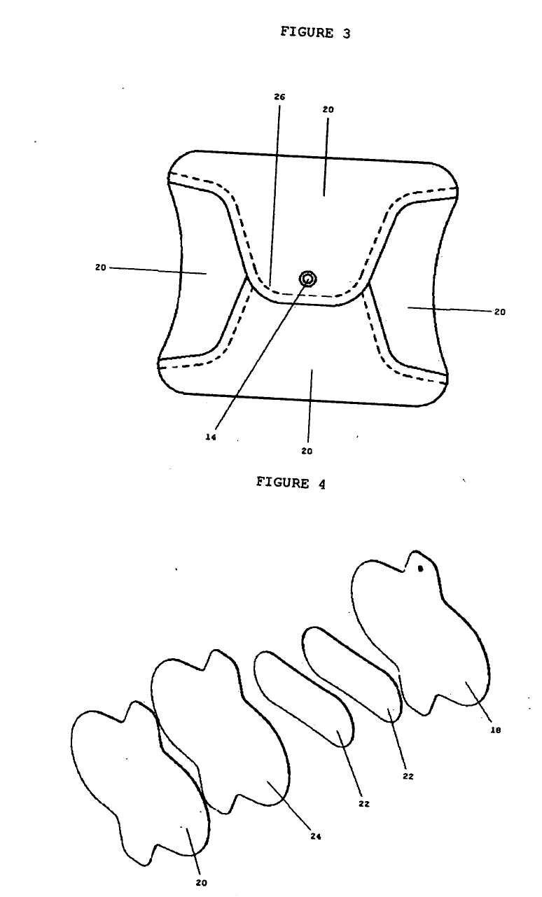

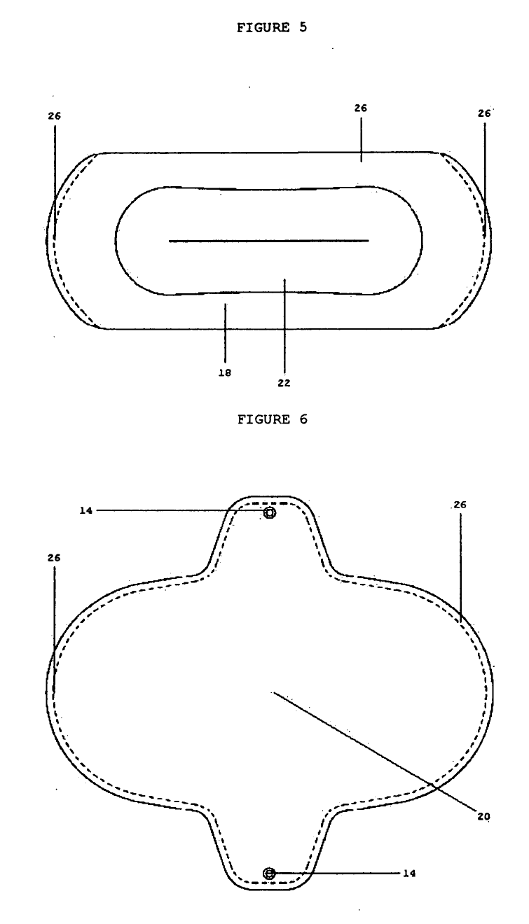

FIGS. 4 illustrates a view of Reusable Cloth Menstrual Pad with Hemp Core 22, folded down and Resin Snap 14 closed into a convenient liquid impervious carry pouch according to the present invention.

The present invention, Cloth Menstrual Pad with Hemp Core 22, generally comprises 5 layers of fabric. The first layer, which is Bodyside Fabric 18, is comprised of either Organic Cotton or nonorganic woven material. Second and third layers are the Hemp Core 22; a very absorbent natural material. Fourth layer is a Leak Guard 24, which is gas permeable, liquid impervious polyurethane laminate Fifth layer is the Backside Fabric 20, which is either organic cotton or nonorganic woven material.

All five layers of fabric are sewn together around the perimeter, and then turned and Top-stitching 26 applied which neatly and smoothly holds all fabric layers together and is esthetically pleasing. The apparatus is designed to attach, by means of Resin Snap 14, around the underside of the users undergarment. Once snapped in place the reusable cloth menstrual pad will thoroughly and discreetly catch menses or urine. The product is practical, comfortable to wear and is easily washed for reuse.

Just as women vary in size and shape the cloth menstrual pads with Hemp Core 22 comes in four sizes, 9 inch, 10.5 inch and 12 inch.

It will also be understood that the device can be used for urine incontinence and as a natural alternative to disposable menstrual pads and urinary incontinence products.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. a cloth menstrual pad with hemp core for comfortably and discreetly collecting menses and urine incontinence in a natural, comfortable and eco-friendly manner, comprising: means for stabilizing pad in it's original position around users undergarment;
means for providing softness and comfort against users body, topically attached to said means for stabilizing pad in its original position around users undergarment;
means for controlling slipping of the pad. Also provided for softness and esthetics;
means for absorption of menses and urine, firstly sewn to said means for providing softness and comfort against users body;
means for ensuring a leakproof device, secondly sewn to said means for absorption of menses and urine, and thirdly sewn to said means for controlling slipping of the pad. Also provided for softness and esthetics; and
means for holding material layers all together.

2. The cloth menstrual pad with hemp core in accordance with claim 1, wherein said means for stabilizing pad in its original position around users undergarment comprises a light weight, resin snap.

3. The cloth menstrual pad with hemp core in accordance with claim 1, wherein said means for providing softness and comfort against users body comprises an organic or nonorganic woven bodyside fabric.

4. The cloth menstrual pad with hemp core in accordance with claim 1, wherein said means for controlling slipping of the pad. Also provided for softness and esthetics comprises an organic or non-organic backside fabric.

5. The cloth menstrual pad with hemp core in accordance with claim 1, wherein said means for absorption of menses and urine comprises a two-layer hemp core aligned centrally, naturally antimicrobial, naturally antibacterial hemp core.

6. The cloth menstrual pad with hemp core in accordance with claim 1, wherein said means for ensuring a leakproof device comprises a liquid impervious polyurethane laminate, layered between the bodyside fabric and the backside fabric leak guard.

7. The cloth menstrual pad with hemp core in accordance with claim 1, wherein said means for holding material layers all together comprises an applied to perimeter of the pad top-stitching.

8. A cloth menstrual pad with hemp core for comfortably and discreetly collecting menses and urine incontinence in a natural, comfortable and eco-friendly manner, comprising: a light weight, resin resin snap, for stabilizing pad in it's original position around users undergarment;
an organic or nonorganic woven bodyside fabric, for providing softness and comfort against users body, topically attached to said Resin Snap;
An organic or non-organic backside fabric, for controlling slipping of the pad. Also provided for softness and esthetics;
a two-layer hemp core aligned centrally, naturally antimicrobial, naturally antibacterial hemp core, for absorption of menses and urine, firstly sewn to said Bodyside Fabric;
a liquid impervious polyurethane laminate, layered between the bodyside fabric and the backside fabric leak guard, for ensuring a leakproof device, secondly sewn to said Hemp Core, and thirdly sewn to said Backside Fabric; and applied to perimeter of the pad top-stitching, for holding material layers all together.

9. The cloth menstrual pad with hemp core as recited in claim 8, wherein said bodyside fabric is natural coloring via Color Grown cotton.

10. The cloth menstrual pad with hemp core as recited in claim 8, wherein said backside fabric is natural coloring via Color Grown cotton.

11. The cloth menstrual pad with hemp core as recited in claim 9, wherein said backside fabric is natural coloring via Color Grown cotton.

12. A cloth menstrual pad with hemp core for comfortably and discreetly collecting menses and urine incontinence in a natural, comfortable and eco-friendly manner, comprising: a light weight, resin snap, for stabilizing pad in it's original position around users undergarment;
an organic or nonorganic woven, natural coloring via color grown cotton bodyside fabric, for providing softness and comfort against users body, topically attached to said Resin Snap;
an organic or non-organic, natural coloring via color grown cotton backside fabric, for controlling slipping of the pad. Also provided for softness and esthetics;
a two-layer hemp core aligned centrally, naturally antimicrobial, naturally antibacterial hemp core, for absorption of menses and urine, firstly sewn to said Bodyside Fabric;
a liquid impervious polyurethane laminate, layered between the bodyside fabric and the backside fabric leak guard, for ensuring a leakproof device, secondly sewn to said Hemp Core, and thirdly sewn to said Backside Fabric; and
an applied to perimeter of the pad topstitching, for holding material layers all together
